(19) United States
(12) Patent Application Publication
(10) Pub. No.: US 2002/0138150 A1
(43) Pub. Date: Sep. 26, 2002

(54) Knee prosthesis

(75) Inventor: Vincent Leclercq, Winterthur (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sulzer Orthopedics, Ltd., Baar (CH)

(21) Appl. No.: 10/043,690

(22) Filed: Jan. 10, 2002

(30) Foreign Application Priority Data
Mar. 26, 2001 (EP) 01810301.0

(51) Int. Cl. A61F 2/38

(52) U.S. Cl. 623/20.35

(57) ABSTRACT

Femur parts of knee prostheses are shown with the invention which have a center part (2) and a condyle part (3) with contact surfaces (4a, 5a, 6a, 6b, 7b, 8a, 9a, 10a, 11a, 11b, 12a, 12b, 13b) with respect to a resectioned femur stump (1), with the center part (2) having guide surfaces (20) for a patella (19) and outer contact surfaces (4a, 5a) which are at an acute angle α≦90° relative to one another, while the condyle part (3) has running surfaces (27, 28) for the articulation movement and outer contact surfaces (6b, 7b) at an acute angle β≦90° relative to one another. The center part (2) and the condyle part (3) can be pushed onto the femur stump separately from one another at push-on directions pivoted with respect to one another in order to allow a greater flexion, with the center line of the angle β being pivoted through an angle of 15°≦γ≦60° with respect to the center line of the angle α.

The invention relates to a knee prosthesis having a femur part which has guide surfaces for a patella and at least one condyle which has outer contact surfaces at an acute angle of α≦90° and whose contact surfaces can be pushed onto resection surfaces at the stump of a femur bone along a straight line in the pivot range of the angle α.

It has previously been usual in a knee prosthesis to push and fasten the condyles onto a pre-worked femur in the form of an upwardly open “U”. For this purpose, the femur stump is brought to the shape of the contact surfaces of the artificial condyle parts in the region of the natural condyles in order to subsequently fasten them. The actual fastening can be carried out by projecting spigots at the contact surfaces, by a wedging of the outer opposing contact surfaces and/or by a fixation of the contact surfaces using bone cement. The condyles contact a meniscus part or a platform and can be pivoted on their bearing surfaces relative to the platform or tibia respectively through a flexion angle during the articulation of the knee joint.

Artificial knee joints are shown in EP-A-0 519 873 in which, depending on the state of the ligaments, a more or less effective lateral guidance of the condyles is also possible during the articulation.

The upper half of the prosthesis has guide surfaces for a patella which is anteriorly arranged, and the patella, which is offset by around 90° with respect to the platform, slides on its own guide surfaces at the upper half of the prosthesis during flexion. The contact surfaces of the upper half of the prosthesis form a “U” shaped opening towards the top, with the patella contacting the front limb and the platform contacting the bottom of this “U” in the extension, whereas the bottom rotates under the patella and the rear limb rotates relative to the platform during flexion. Both forces from the patella from the front and forces from the rear occur at the femur stump during flexion and are taken up by opposing resection surfaces which are approximately perpendicular to one another. In most upper parts of a prosthesis, the associated outer contact surfaces have a small angle relative to one another so that a tight fit relative to the prosthesis stump is achieved by the wedging action when pushed on. The flexion angle obtainable with such a prosthesis is insufficient for many inhabitants in Asian countries since they are naturally used to living with a larger flexion angle and bring a knee prosthesis into flexion angles which are not suitable for it.

It is the object of the invention to provide design forms which allow large flexion angles with an open joint. This object is satisfied in that the femur part has a center part with guide surfaces for the patella and a condyle part which can be pushed on separately therefrom, which has outer contact surfaces at an acute angle β≦90° and which can be pushed onto resection surfaces at the stump of the femur along a straight line in the pivot range of the angle β, with the centre line of the angle β at the condyle part being pivoted through an angle 15°≦γ≦60° relative to the centre line of the of the angle a at the centre part in order to allow a greater flexion.

The advantage of the invention lies in the fact that the forces which are transmitted to the prosthesis stump by the upper part of the prosthesis are compressive forces even with a large flexion. No shear stresses occur between the contact surfaces and the resection surfaces which are opposite to the direction of pushing on. Since the upper part of the prosthesis is divided into a centre part and a condyle part, whose dividing joint extends such that the guide surfaces for the patella are arranged in the centre part and since bearing surfaces are arranged with respect to the platform on the condyle part, these surfaces can be made independently of one another and be pushed onto the femur stump independently of one another in pushing directions which are pivoted independently of one another. Contact surfaces of the centre part and the condyle part can jointly contact one resection surface despite the different pushing directions. The pushing directions are pivoted, for example, through an angle γ of between 15° and 60° in order to allow a large flexion angle δ. Flexion angles δ of more than 120°, for example 160°, are possible. The condyles can be connected by a yoke or formed singly. When pushed on, the condyles are aligned with respect to the centre part by the resection surfaces, that is via the femur stump. In addition, the centre part and the condyle parts can be movably guided with respect to one another in sagittal planes in order to obtain similar force exertion points of the patella and the platform at the femur stump as in a natural knee joint. However, the possibility also exists to connect the centre part and the condyles by means of a coupling member in the pushed-on state.

Individual contact surfaces can have pockets in order to introduce a minimum amount of bone cement for a fastening with bone cement. Individual contact areas, in particular the outer contact areas, can have a serrated anchoring structure for a primary anchoring. Equally, individual spigots, which extend in the pushing direction and for which bores are provided in the pushing direction, can improve the primary anchoring. The contact surfaces not wetted with bone cement can be coated with a layer which promotes bone growth.

Contact surfaces wetted by bone cement can be coated with an adhesion promoting agent. Such a layer can, for example, be built up of a titanal alcoholate and an alkoxy silane.

During flexion, the engaged radius of curvature of the condyles can reduce continuously or in stages in order to allow a lateral deflection of the condyles as the flexion increases.

The invention is described in the following by means of embodiments. There are shown:

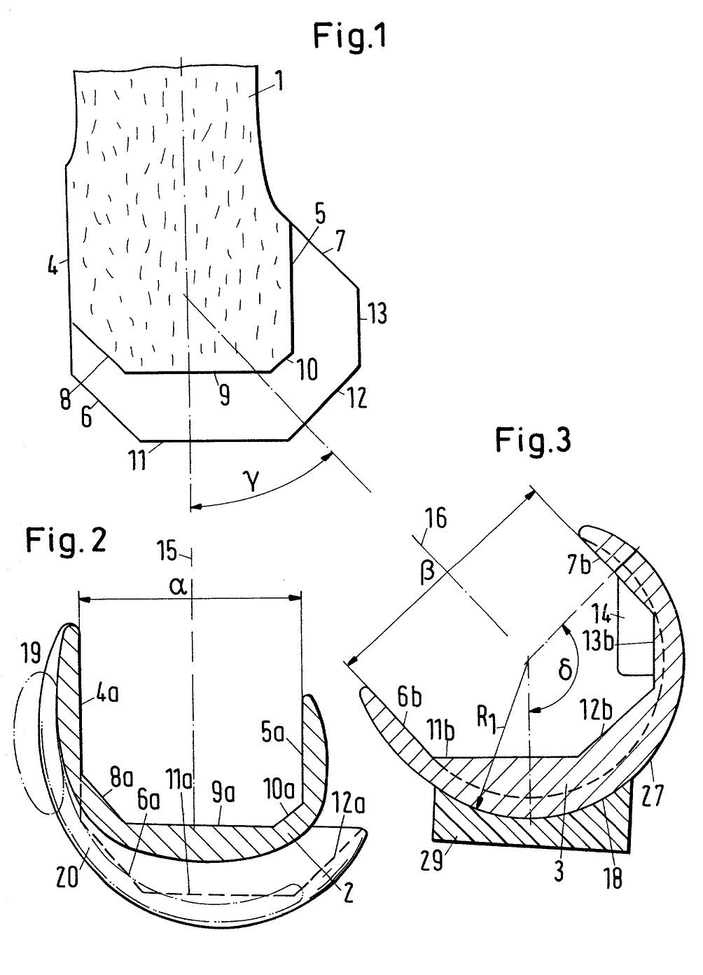

FIG. 1: a femur stump with pre-worked resection surfaces, in schematic form, onto which, in accordance with

FIG. 2: a schematically shown centre part can be pushed along a centre line and, in accordance with

FIG. 3: a schematically shown condyle part can be pushed along a centre line pivoted through an angle γ;

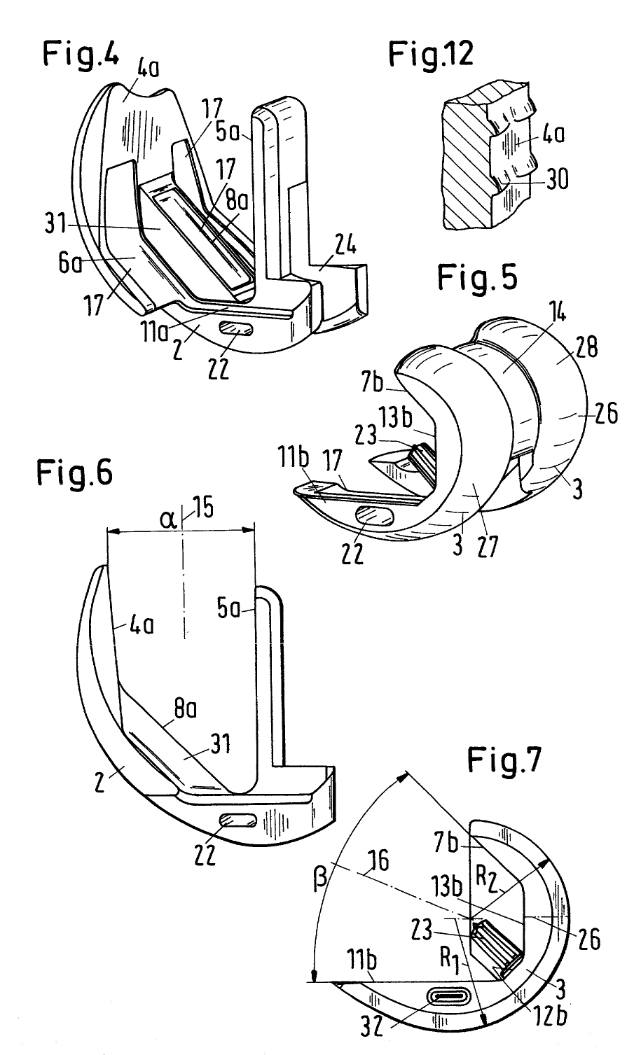

FIG. 4: a view of a further example of a centre part in schematic form;

FIG. 5: a condyle part matching FIG. 4 with a yoke, in schematic form;

FIG. 6: a side view of FIG. 4 in schematic form;

FIG. 7: a side view of FIG. 5 in schematic form;

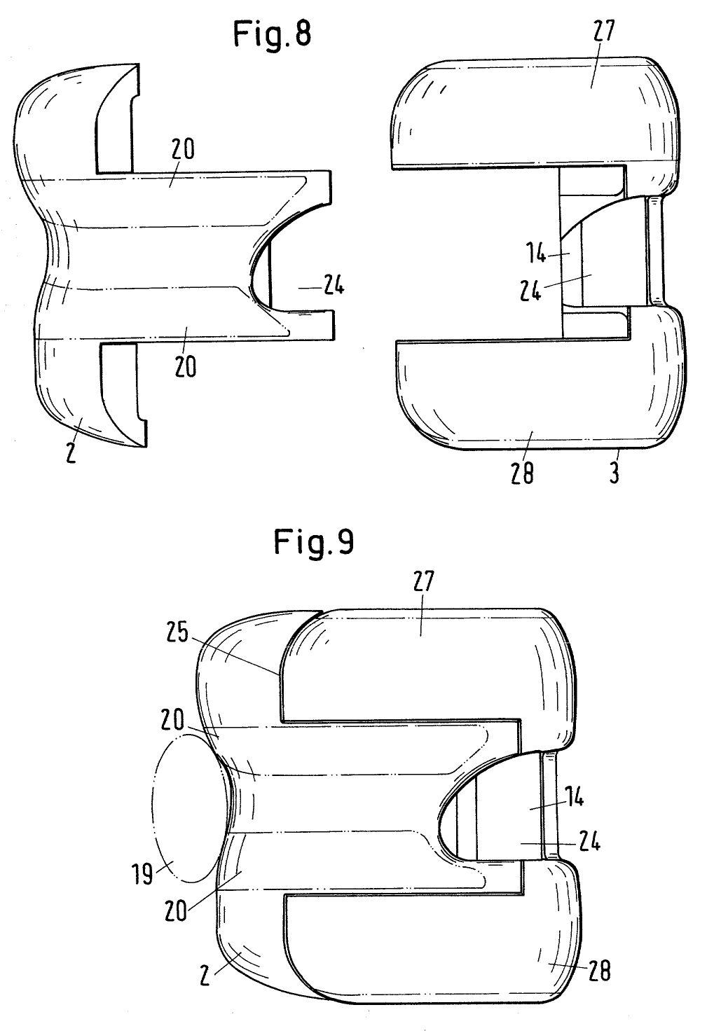

FIG. 8 a view from below of FIGS. 4 and 5 prior to the pushing on, in schematic form;

FIG. 9 the front and condyle part of FIG. 8 after the pushing on, in schematic form;

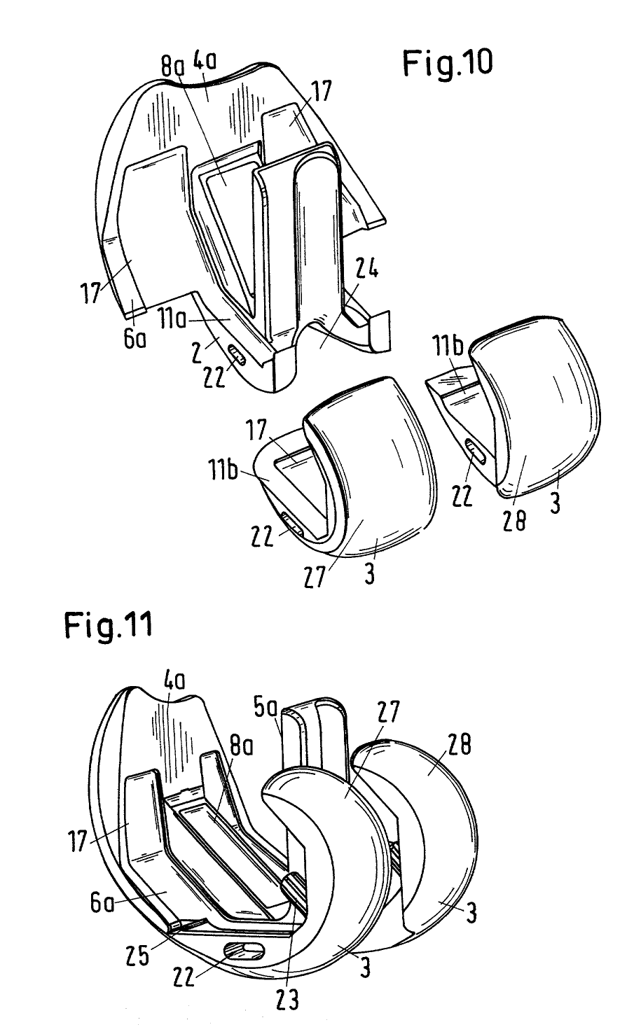

FIG. 10 a view of a centre part having two condyles separate from one another, prior to pushing on, in schematic form;

FIG. 11 the parts of FIG. 10 after pushing on at a somewhat different angle of view, in schematic form; and

FIG. 12 a n enlarged section of an outer contact surface with a serrated design for the primary anchoring, in schematic form.

Femur parts of knee prostheses are shown in the figures which have a centre part 2 and a condyle part 3 with contact surfaces 4a, 5a, 6a, 6b, 7b, 8a, 9a, 10a, 11a, 11b, 12a, 12b, 13b with respect to a resectioned femur stump 1, with the centre part 2 having guide surfaces 20 for a patella 19 and outer contact surfaces 4a, 5a which are at an acute angle α≦90° to one another, while the condyle part 3 has running surfaces 27, 28 for the articulation movement and outer contact surfaces 6b, 7b at an acute angle β≦90° to one another. The centre part 2 and the condyle part 3 can be pushed onto the femur stump separately from one another in push-on directions pivoted with respect to one another in order to allow a greater flexion, with the centre line of the angle β being pivoted by an angle 15°≦γ≦60° with respect to the centre line of the angle α.

The same reference numerals are used for the same functions in the following.

In the example of FIGS. 1, 2 and 3, resection areas 4, 6, 11, 12, 12, 13, 7 are provided in the region of the condyles and resection areas 4, 8, 9, 10, 5 in the region between the condyles. The resection areas 4 and 5 and the outer contact surfaces 4a, 5a of the centre part 2 which contact them are almost parallel to one another at an acute angle. The contact surfaces 8a, 9a, 10a of the centre part are supported between the condyles and further contact surfaces 6a, 11a 12a thereof are supported on resection areas in the region of the prior, natural condyles, with these resection areas also being occupied by contact surfaces 6b, 11b, 12b of the condyle part 3 in order to achieve centering in the groove between the condyles and to also give support to the guide surfaces 20 for the patella 19 at full flexion. The centre part can be pushed on from below along the centre line 15 of the angle α, while the push-on direction and the centre line 16 of the angle β is pivoted through an angle γ of 45° in order to produce an outer contact surface 7b, even at a larger flexion angle δ, for example δ>120°, at which the contact force presses the area 7b into the stump or at most perpendicular to the area 7b. As can be seen from FIG. 3, the running surface 27 of the condyle part 3 and the running surface 18 of a meniscus part 18 have the same radius of curvature R₁.

A further example is shown in the FIGS. 4, 5, 6, 7, 8, 9. The guide surfaces 20 for the patella 19 and the running surfaces 27, 28 also have to be extended here in the flexion direction due to the extended flexion angle. The centre part 2 has contact surfaces 4a, 6a, 8a, 11a, 5a and lateral centering surfaces 31. The condyle part 2 has a yoke 14 which connects the actual condyles. A separating joint 25 between the centre part 2 and the condyle part 3 is disposed (FIGS. 8, 9) such that the guide surfaces 20 for the patella 19 and the running surfaces 27, 28 of the condyles are not interrupted. A recess 24 is provided for cruciate ligaments. A part of the contact surfaces 4a, 6a, 8a, 11a has pockets 17 which are filled with bone cement during the pushing on in order to allow a minimum layer thickness of bone cement in certain regions. The pockets 17 and contact surfaces can also be pre-cemented with an adhesion agent and a thin layer of bone cement.

In accordance with the acute angle a in FIG. 6, the pushing on of the centre part 2 has to be carried out roughly in the direction of the centre line 15, whereas with the condyle part with a less acute angle β, a greater deviation of the push-on direction from the centre line of the angle β is possible. The push-on direction here, which has to be within the angle β, is determined by the direction of the spigots 23. A generatrix is indicated for the contour of the condyle running surfaces 27, 28 in FIGS. 5 and 7. As the flexion increases, the radius of curvature R₁ of the running surfaces 27, 28 changes into a smaller radius of curvature R₂.

The centre part 2 and the condyle part 3 have bores 22 so that they can be connected to a coupling piece in the form of a pin 32 after being pushed onto the femur stump. The femur stump is simultaneously trapped by this connection since it is surrounded by contact surfaces at an enveloping angle of more than 180°.

The section of the contact surface 4a in FIG. 12 shows that it can be meaningful, in the event of outer contact surfaces which are at a very acute angle to one another, such as for example the surfaces 4a and Sa in FIG. 6, to provide this surface with a serration which allows a good primary anchoring and secures the centre part 2 until the condyle part 3 has also been pushed on.

An arrangement is shown for the example of FIGS. 10 and 11, in which two separate condyle parts 3 with running surfaces 27, 28 can be pushed on and can be connected to the centre part 2 by a coupling piece, for example a pin or a screw, via bores 22. If the cruciate ligaments at the knee allow, these two condyle parts 3 can also be introduced from the side since they are not fixedly connected to a yoke in order then—depending on how long the journals 23 are—to find their anchorage in the push-on direction of the journals. As soon as a condyle part 3 is connected to the centre part 2, the femur stump is also surrounded at an enveloping angle of more than 180° here and a flexion angle of more than 120° is possible without risk.

1. A knee prosthesis having a femur part which has guide surfaces (20) for a patella (19) and at least one condyle (18), which has outer contact surfaces (4a, 5a) at an acute angle α≦90° and whose contact surfaces can be pushed onto resection areas (4, 5, 6, 8, 9, 10, 11, 12) at the stump of a femur bone (1) along a straight line in the pivot range of the angle α, characterised in that the femur part has a centre part (2) with guide surfaces (20) for the patella (19) and a condyle part (3) which can be pushed on separately, which has outer contact surfaces (6b, 7b) at an acute angle β≦90° and which can be pushed onto resection areas (6, 7, 11, 12, 13) at the stump of the femur bone along a straight line in the pivot range of the angle β, with the centre line (16) of the angle β at the condyle part (3) being pivoted through an angle of 15°≦γ≦60° with respect to the centre line (15) of the angle α at the centre part (2) in order to allow a greater flexion.

2. A knee prosthesis in accordance with claim 1, characterised in that the condyle part (3) has a flexion angle δ of more than 120°.

3. A knee prosthesis in accordance with claim 1 or claim 2, characterised in that the condyle part (3) has two condyles (3) each outwardly disposed with respect to the centre part (2) which are connected via a yoke (14).

4. A knee prosthesis in accordance with any of claims 1 to 3, characterised in that the centre part (2) and the condyle part (3) are connectable to one another in the pushed-on state by a coupling member (21).

5. A knee prosthesis in accordance with claim 4, characterised in that the coupling member (21) consists of a pin inserted transversely through separating surfaces or of a transversely throughgoing screw connection.

6. A knee prosthesis in accordance with anyone of claims 1 to 5, characterised in that the angle γ is between 40° and 50°.

7. A knee prosthesis in accordance with any of claims 1 to 6, characterised in that individual contact surfaces (4a, 5a, 6a, 6b, 7b, 8a, 9a, 10a, 11a, 11b, 12a, 12b, 13b) have pockets (17) in order to introduce a minimum quantity of bone cement for a fastening with bone cement.

8. A knee prosthesis in accordance with of claims 1 to 6, characterised in that individual contact surfaces (4a, 5a, 6a, 6b, 7b, 8a, 9a, 10a, 11a, 11b, 12a, 12b, 13b) have a serrated anchoring structure (30) for a primary anchoring.

9. A knee prosthesis in accordance with any of claims 1 to 6, characterised in that individual contact surfaces (4a, 5a, 6a, 6b, 7b, 8a, 9a, 10a, 11a, 11b, 12a, 12b, 13b) are covered with a coating which promotes bone growth or enhances the adhesion of bone cement.

10. A knee prosthesis in accordance with claim 9, characterised in that the coating contains substances such as hydroxyl appatite or is made up of a titanal alcoholate and an alkoxy silane.

11. A knee prosthesis in accordance with any of claims 1 to 10, characterised in that the radius of curvature R₁ of the condyles (3) are reduced at least once in the contact region from the transition of the extension to the full flexion.

12. A knee prosthesis in accordance with any of claims 1 to 11, characterised in that the angle α or β is less than 10°.

13. A knee prosthesis in accordance with any of claims 1 to 11, characterised in that the angle α or β is less than 5°.

14. A knee prosthesis in accordance with any of claims 1 to 13, characterised in that spigots (23) are attached to contact surfaces (12b) in the direction of pushing on which facilitate centering and primary anchoring.
